(19) United States
(12) Patent Application Publication
(10) Pub. No.: US 2020/0281845 A1
(43) Pub. Date: Sep. 10, 2020

(54) COMPOSITIONS COMPRISING LESPEDEZA PLANT EXTRACT

(71) Applicant: Clariant International Ltd., Muttenz (CH)

(72) Inventors: Mi Ji YEOM, Gyeonggi-do (KR); Eun Sun JUNG, Gyeonggi-do (KR); Deok Hoon PARK, Gyeonggi-do (KR); Mathilde FRECHET, Toulouse Cedex 1 (FR); Hanane CHAJRA, Toulouse Cedex 1 (FR)

(73) Assignee: Clariant International Ltd., Muttenz (CH)

(21) Appl. No.: 16/645,878

(22) PCT Filed: Apr. 9, 2018
(86) PCT No.: PCT/EP2018/058969

§ 371 Date: Mar. 10, 2020

(30) Foreign Application Priority Data
Sep. 13, 2017 (KR) 10-2017-0117372

(51) Int. Cl. A61K 8/9789; A61K 36/48; A61Q 19/08

(52) U.S. Cl. CPC A61K 8/9789; A61K 9/0014; A61Q 19/08; A61K 36/48

(57) ABSTRACT

The present invention relates to a composition for use in preventing or alleviating circadian rhythm disorder in a mammal, the composition comprising an extract from a Lespedeza sp. plant.

### DESCRIPTION OF INVENTION

A composition for use in preventing or alleviating circadian rhythm disorder in a mammal, especially mammalian skin, a method of preventing or alleviating circadian rhythm disorder in a mammal, especially in mammalian skin and the use of a composition comprising an extract from an Lespedeza sp. plant for a cosmetic, non-therapeutic treatment to increase the radiance of the skin or to improve the complexion of skin or to enhance skin biorhythms.

### TECHNICAL FIELD

The present invention relates to for preventing or alleviating circadian rhythm disorders using a Lespedeza plant extract as an active ingredient, more specifically, cosmetics, pharmaceutical composition and food composition for preventing or alleviating circadian rhythm disorders comprising a Lespedeza plant extract that is effective for enhancing circadian rhythm.

### BACKGROUND ART

Most living organisms including mammals adapt to the day and night changes and a periodic pattern of 24 hours, and this is called the “circadian rhythm”. As such, living organisms have developed a periodic and intrinsic circadian clock in order to adapt to repetitive changes of external environment. That is, an adaptation that helps the biological process to occur at an appropriate moment adjusting to the environmental changes. The circadian clock is synchronized by external signals such as light, temperature, and food intake etc., and after synchronization, it can continuously retain the rhythm of 24 hours by itself without any external signals. The self-sustaining circadian clock, allows living organisms to predict and cope with the external environments; they have developed this ability during the evolutionary process.

The circadian clock can be mainly divided into 2 parts, that is, a central clock located in the suprachiasmatic nucleus of hypothalamus and a peripheral clock located in various peripheral tissues in the body. The central clock controls the peripheral clock through neural signals, hormones, and body temperature. The peripheral clock located in the liver, kidney, and pancreas is not only regulated by a central clock but also responds to the external or internal signals by itself.

The circadian clock consists of Transcription-Translation Loop (TTL) of specific circadian clock proteins at a molecular level. Among the main circadian clock proteins, Clock and Bmal1 have a form of heterodimer, and they induce the expression of Per, Cry, Ppar, Rev-Erb, which are the circadian clock-related genes with E-box promoter. On the other hand, the expressed Per and Cry have a form of heterodimer, and they establish a negative feedback loop by suppressing the activation of Clock/Bmal1. Through the negative feedback loop of transcription-translation of circadian clock proteins, expression of circadian clock-related genes follows a periodic rhythm. In addition, the expression rhythm of the 24-hour cycle is established by modulating optimal time delay and advancing through post-translational modification.

The circadian clock proteins have increased expression and a more activated state at a certain point during the day and regulate the expression and activation of genes and proteins involved in various biological processes in a 24-hour cycle. Through these, the circadian clock regulates the level of biological responses according to the external environment.

Since the circadian clock is a universal regulator that regulates the physiology, metabolism, and behaviour in general, disturbance of daily circadian rhythm is not only a common cause of sleep disorders and fatigue syndrome but also metabolic diseases, such as obesity and diabetes, cardiovascular diseases, various tumours, rheumatism, Dementia, and psychiatric disorders. In fact, the World Health Organization (WHO) stated in 2009 that circadian rhythm disturbance could be a major risk factor for the development of cancer and metabolic diseases.

The discovery of molecular circadian clock genes and the development of various mutant mouse models have provided new insights into the research related to physiological problems caused by circadian rhythm disturbances and pathogenesis of major diseases associated with circadian rhythms.

Circadian rhythm sleep disorders are sleep disorders in which the sleep-wake rhythm appears abnormal due to genetic or environmental disturbances in the circadian clock. If the circadian status of sleep-wake rhythm is abnormally slow or fast, circadian rhythm sleep disorders can occur including sleep fragmentation caused by disruption of periodicity and a decrease of sleep duration, and the fragmentation of intrinsic circadian rhythms and the sleep cycle caused by environmental factors (time difference, shift work, etc.). In particular, many patients with insomnia and hypersomnia (10-40%) are expected to have circadian rhythm sleep disorder, and recently these 3 major types of sleep disorder have been generally classified as circadian rhythm disorders, because they are commonly associated with the disturbance of the circadian rhythms. Circadian rhythm sleep disorder patients have difficulty in falling asleep or waking up at socially required times, and thus demonstrate symptoms caused by a lack of sleep. However, following the discovery of the molecular circadian clock, various genetic studies have identified that there is a genetic circadian rhythm sleep disorder caused by a mutation in PERs or CRYs among the circadian clock genes (Sehgal and Mignot, Cell. 2011 Jul. 22; 146(2): 194-207). The above genetic studies commonly identified that the mutant mice with deletion of these circadian clock genes have shown abnormal cycle or status in sleep-wake rhythm. In addition, mutant mice lacking these circadian clock genes tend to have increased non-REM (rapid eye movement) levels (NREM S) and increased intensity of delta waves compared to normal mice. In contrast to this, it was reported that in the case of animals whose circadian clock is disturbed by destroying SCN of the hypothalamus, where the central clock exists, there was no change in the total sleep time, but the sleep pattern was fragmented and the intensity of the delta wave also seemed to decrease. This suggests that circadian clock genes have an important role in regulating sleep homeostasis, especially sleep quality, in addition to maintaining the circadian rhythm.

Skin cells including keratinocytes, melanocytes, fibroblasts, also have an independent circadian clock system. The circadian clock system also affects the function of skin by expressing circadian clock proteins not only in the cultured human skin cells, but also in human skin tissue. Skin is easily exposed to external environmental factors such as light, heat, moisture, UV rays, pathogenic bacterium etc., and these environmental factors change in a daily cycle. In order to adapt to the environment, skin activates its inherent circadian clock, and regulates various physiological functions. It is known that proliferation and division of skin cells, hydration and trans-epidermal water loss (TEWL), capillary blood flow, sebum creation, temperature, surface pH, wrinkle formation change in a 24-hour cycle. For instance, skin cell proliferation and differentiation occur through a series of processes in a daily cycle. Skin cell differentiation mostly occurs between late night and early morning, and during the day time when it is easy to be exposed to UV rays, DNA damage protection mechanisms mostly occur, followed by cell division. By separating these processes in time, it is possible to protect skin cells from harmful environments. If the rhythm of this process breaks down, physiological responses cannot occur at an appropriate time, and this will disturb the homeostasis of the skin, and cause skin damage.

The circadian clock can be disturbed by external environmental conditions, which disturb the circadian cycle, or its rhythmicity can be reduced by endogenous aging. Rhythmicity can be defined as an amplitude between a peak and a trough in a sinusoidal pattern, and the reduction in rhythmicity means that the amplitude between a peak and a trough has been reduced. The rhythmicity of the circadian clock can be weakened by aging or degenerative diseases such as Alzheimer's disease. In general, aging causes a reduction in the neurological activity of the pituitary gland and the sensitivity to light, which can synchronize the circadian rhythm, and with fewer daytime activities, the circadian rhythm becomes weaker, resulting in reduced circadian rhythm in body temperature changes and hormones (melatonin, cortisol etc.), which, in turn may cause circadian rhythm sleep disorder (Akerstedt T, et al., Sleep Med Clin. 2009 Jun. 1; 4(2):257-271). In addition, the circadian rhythm may be disturbed by external environmental stress, for instance, it may be disturbed by the blue light emitted from the electronic device or artificial light and can be weakened by environmental hormones.

Recently, a search for materials targeting the circadian clock has been conducted with the intention of enhancing biorhythms.

The Lespedeza genus includes Lespedeza bicolor Turcz, Lespedeza cuneata G. Don, Lespedeza cyrtobotrya Miq, Lespedeza juncea (L. f.) Pers, Lespedeza capitata and Lespedeza maximowiczii C. K. Schneid, etc.

Lespedeza bicolor Turcz is a deciduous broad-leaved shrub belonging to the bean and Lespedeza genus. Leaves of Lespedeza bicolor Turcz contain alkaloids, flavonoids, and ascorbic acid, the shells contain tannins, and the shell, stems and leaves contain saponins. The roots contain several types of alkaloids. The roots of Lespedeza bicolor Turcz are traditionally well known to be effective against paralysis, bruises, leukorrhea, boils, Rheumatoid arthritis, and lumbago. The leaves and stems of Lespedeza bicolor Turcz are known to treat headaches, coughs caused by the heat in lung, heart diseases, and whooping cough.

Lespedeza cuneata G. Don is a perennial plant belonging to the bean and Lespedeza genus. The leaves, stems, and roots of Lespedeza cuneata G. Don contain flavonoids, pinitol, phenols, tannins, beta-sitosterol etc., and these substances are known to be effective for suppressing Staphylococcus aureus, Pneumococcus, Alpha Streptococcus and Qatar Strain.

Lespedeza capitata, known as roundhead bush clover grows wild in Asia. It has been used for its traditional medicinal properties as a depurative and a tonic in Asia and the United States. It is known to contain flavonoids.

However, until now there has been little research on the effects of Lespedeza plant extracts in modulating biorhythm, and its therapeutic effects for preventing or alleviating circadian rhythm disorders.

The present inventors have been researching to find a material for restoring and enhancing biorhythmicity reduced by the external environment, and have identified biorhythm-enhancing effects in Lespedeza plants such as Lespedeza capitata, Lespedeza bicolor Turcz, Lespedeza cuneata G. Don, and Lespedeza maximowiczii C. K. Schneid.

It is against this background that the present invention has been devised.

### SUMMARY OF INVENTION

According to a first aspect of the invention, a composition is provided for use in preventing or alleviating circadian rhythm disorder in a mammal, the composition comprising an extract from a Lespedeza sp. plant.

According to a second aspect of the invention, a composition is provided for use in preventing or alleviating circadian rhythm disorder in mammalian skin, the composition comprising an extract from a Lespedeza sp. plant.

According to a third aspect of the invention, a method of preventing or alleviating circadian rhythm disorder in a mammal is provided, the method comprising administering to a mammal in need thereof a composition comprising an extract from a Lespedeza sp. plant.

According to a fourth aspect of the invention, a method of preventing or alleviating circadian rhythm disorder in mammalian skin is provided, the method comprising administering to the skin of a mammal in need thereof a composition comprising an extract from a Lespedeza sp. plant.

According to a fifth aspect of the invention, the use is provided of a composition comprising an extract from a Lespedeza sp. plant for a cosmetic, non-therapeutic treatment to increase the radiance of the skin.

According to a sixth aspect of the invention, the use is provided of a composition comprising an extract from a Lespedeza sp. plant for a cosmetic, non-therapeutic treatment to improve the complexion of the skin.

According to a seventh aspect of the invention, the use is provided of a composition comprising an extract from a Lespedeza sp. plant for a cosmetic, non-therapeutic treatment to enhance skin biorhythms.

Also, the present invention also provides a food composition for preventing or alleviating circadian rhythm disorder, comprising the Lespedeza plant extract as an active ingredient.

The term “circadian rhythm disorder” refers to the disorder caused by a disturbance of biorhythms, and the disturbance of biorhythms is caused by reduction in the expression of circadian clock genes.

In the present invention, the Lespedeza plant is one or more Lespedeza plant selected from a group consisting of Lespedeza capitata, Lespedeza bicolor Turcz, Lespedeza cuneata G. Don and Lespedeza maximowiczii C. K. Schneid.

In the present invention, the parts of Lespedeza plants which may be used are the leaves, flower, roots, stem, and seed, but preferably the stem and/or root of Lespedeza plants.

The term in the present invention “extract” refers to a formulation prepared by extracting a herbal medicine with an appropriate leaching solution and concentrating the leaching solution, and it may include, but is not limited to, an extract obtained by the extraction method, a diluted or concentrated form of the extract, a dried product obtained by drying the extract, or a solution emulsion or suspension thereof in partially purified water or purified water.

In the present invention, Lespedeza plant extract may be produced using common extraction, isolation and purification methods known in the art. Examples of such extraction methods include, but are not limited to, boiling extraction, hot water extraction, cold extraction, reflux cooling extraction and sonication extraction.

In addition, the Lespedeza plant extract may be obtained by a conventional purification process in addition to the above extraction methods that use extraction solvent. For example, the Lespedeza plant extract may be obtained in a fraction produced by conducting other purification methods including separation through an ultrafiltration membrane with a constant molecular weight cut-off value, or separation through various chromatographic methods (made for separation by size, charge, hydrophobicity or affinity).

According to an exemplary embodiment of the present invention, the Lespedeza plant extract may be prepared by drying the stem or roots of each Lespedeza plant, pulverizing them, and then extracting them with water, organic solvent or a mixture thereof, followed by filtration under reduced pressure. The organic solvent may comprise methanol, ethanol, propanol or butanol, and preferably comprises ethanol, more preferably at least 70% ethanol.

In the present invention, the “active ingredient” refers to a component that exhibits the desired activity alone or exhibits the activity together with a carrier that is itself inactive.

The composition of the present invention may comprise from 0.00001 to 15% by weight of Lespedeza plant extract, more preferably from 0.0001 to 10% by weight, and most preferably from 0.001 to 5% by weight, based on the total weight of the composition. If the content of Lespedeza plant extract is less than 0.00001% by weight, the target effect of the present invention, which is the preventative or alleviating effects against circadian rhythm disorder, may not be obtained, and if the content is above 15% by weight, the resulting effect may not be proportional to the increase of the content and thus it may be inefficient, and the stability of formulation may not be ensured.

The composition of the present invention comprising Lespedeza plant extract is effective for preventing or alleviating circadian rhythm disorder.

In the present specification, the term “subject” refers to mammals including humans, monkeys, cows, horses, pigs, sheep, dogs, cats, rats, mice, and chimpanzees wherein the circadian rhythm is weakened or disturbed by, for instance, intrinsic aging or jet lag caused by travelling, UV rays, artificial light, especially blue light, exposure, environmental pollutants, environmental stress such as chemicals or smoking etc. and such disturbance in the circadian rhythm is to be prevented or treated.

In the present specification, the term “prevention” refers to inhibiting the occurrence of a disease or disorder in an individual who has not been diagnosed with them, but is likely susceptible to such disease or disorder.

In the present specification, the term “alleviation” or “treatment” refers to (a) suppression of the development of a disease or disorder in an individual, or (b) removal of a disease or disorder.

In the present specification, the term “administration” refers to introducing the desired substance into the subject in a suitable manner, and the route of administration of the composition of the present invention may be either oral or parenteral through any conventional routes as long as the composition can reach the target tissue. Also, the composition of the present invention may be administered by any device capable of moving the active substance to a target subject, for example, a cell.

The term used in the present specification “circadian rhythm” refers to a periodicity of approximately 24 hours that living organisms including mammals acquired by adapting to the day and night transitions formed by the rotation of the Earth.

According to the exemplary embodiment of the present invention, the present invention provides a composition comprising Lespedeza plant extract as an active ingredient.

The composition of the present invention may contain, in addition to the Lespedeza plant extract as an active ingredient, conventional additives and carriers, such as antioxidants, stabilizers, solubilizers, vitamins, pigments and perfumes.

The composition of the present invention can be prepared into any product form conventionally produced in the art, including, but not limited to, a solution, a suspension, an emulsion, a paste, a gel, a cream, a lotion, a powder, a soap, a surfactant-containing cleanser, an oil, a powder foundation, an emulsion foundation, a wax foundation and a spray. More specifically, it can be formulated into a nourishing cream, an astringent toner, a soft toner, a lotion, an essence, a nourishing gel or a massage cream.

If the formulation of the present invention is a paste, cream or gel, the carrier component may comprise animal oil, vegetable oil, wax, paraffin, starch, tragacanth gum, cellulose derivatives, polyethylene glycol, silicon, bentonite, silica, talc, zinc oxide or mixtures thereof.

If the formulation of the present invention is a powder or spray, the carrier component may comprise talc, silica, aluminium hydroxide, calcium silicate, polyamide powder or mixtures thereof, and if the formulation is a spray, then it may contain propellants such as chlorofluorohydrocarbons, propane/butane or dimethyl ether.

If the formulation of the present invention is a solution or emulsion, a carrier component may comprise a solvent, solubilizing agent or emulsifying agent, for instance water, ethanol, isopropanol, ethyl carbonate, ethyl acetate, benzyl alcohol, benzyl benzoate, propylene glycol, 1.3-butyl glycol oil, glycerol aliphatic esters, polyethylene glycol sorbitan fatty acid esters or mixtures thereof.

If the formulation of the present invention is a suspension, a carrier component may comprise a liquid diluent such as water, ethanol or propylene glycol, or mixtures thereof, a suspension such as ethoxylated isostearyl alcohol, polyoxyethylene sorbitol ester and polyoxyethylene sorbitan ester, microcrystalline cellulose, aluminium metahydroxide, bentonite, agar or mixtures thereof.

If the formulation of the present invention is surfactant-containing cleanser, a carrier component may comprise aliphatic alcohol sulphate, aliphatic alcohol ether sulphate, sulfosuccinic acid monoester, isethionate, imidazolinium derivative, methyltaurate, sarcosinate, fatty acid amide ether sulphates, alkylamidobetaines, polyunsaturated alcohols, fatty acid glycerides, fatty acid diethanolamides, vegetable oils, lanolin derivatives, ethoxylated glycerol fatty acid esters or mixtures thereof.

According to one embodiment of the present invention, the present invention provides a pharmaceutical composition comprising a Lespedeza plant extract as an active ingredient.

The pharmaceutical composition of the present invention comprises a pharmaceutically acceptable carrier in addition to the Lespedeza plant extract. The pharmaceutically acceptable carrier contained in the pharmaceutical composition of the present may be as conventionally used and may comprise lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil or mixtures thereof, but is not limited thereto. The pharmaceutical composition of the present invention may additionally comprise other ingredients such as lubricants, humectants, sweeting agents, flavours, emulsifiers, suspending agents, preservatives and mixtures thereof. A suitable pharmaceutically-acceptable carrier and formulation are described in detail in Remington's Pharmaceutical Sciences (19th ed., 1995).

The pharmaceutical composition of the present invention may be administered orally or parenterally, and is preferably applied by parenteral administration, and more preferably by topical application.

The appropriate dosage of the pharmaceutical composition of the present invention may vary depending on such factors as the formulation method, administration method, age of patient, body weight, sex, pathological condition, food, administration time, administration route, excretion rate and response susceptibility. The dosage of the pharmaceutical composition of the present invention is, on an adult basis, within the range of 0.001-100 mg/kg. If the composition is an external preparation, it is preferable to apply the composition in an amount of 1.0 to 3.0 ml on an adult basis once to five times a day for one month or longer, although it may instead be more or less.

The pharmaceutical composition of the present invention may be prepared according to methods known a person having ordinary skill in the art, and may be formulated in unit dose form or multi-dose container using a pharmaceutically acceptable carrier and/or excipient. The formulations may be in the form of solutions, suspensions, syrups or emulsions in oils or aqueous media, or in the form of powders, granules, tablets or capsules, and may additionally contain dispersing or stabilizing agents.

In addition, according to one embodiment of the present invention, the present invention provides a food composition comprising Lespedeza plant extract as an active ingredient.

The food composition of the present invention may further comprise not only a Lespedeza plant extract as an active ingredient, but one or more components that are ordinarily added in the production of food such as, protein, carbohydrate, fat, nutrients, seasoning agent and flavouring agent.

Examples of carbohydrates include monosaccharides, such as, glucose, and fructose; disaccharides, such as maltose, sucrose, oligosaccharides; and polysaccharides such as dextrin, common sugars such as cyclodextrin etc., and sugar alcohols such as xylitol, sorbitol, and erythritol. A flavouring agent can be natural flavouring agent [tau martin, stevia extract (e.g. rebaudioside A, glycyrrhizin)] a synthetic flavouring agent (saccharin, aspartame) or mixtures thereof.

If the food composition of the present invention is prepared as a drink, other components may be added, in addition to Lespedeza plant extract, such as citric acid, liquid fructose, sugar, glucose, acetic acid, malic acid, juice, mulberry extract, jujube extract, liquorice extract and mixtures thereof.

### Advantageous Effects of Invention

As described above, the composition of the present invention comprising Lespedeza plant extract enhances the circadian clock rhythmicity, and through such effects, it can be effectively used as a composition capable of alleviating, preventing or treating skin damage caused by a biorhythm disorder. In addition, the Lespedeza plant extract is a plant-derived chemical substance and is safe and has little side effects on human body, and thus can be safely used in cosmetic, pharmaceutical and food compositions.

### BRIEF DESCRIPTION OF DRAWINGS

FIGS. 1.1 to 1.4 are graphs demonstrating the biorhythm-enhancing effect of extracts of Lespedeza bicolor Turcz, Lespedeza cuneata G. Don, Lespedeza maximowiczii C. K. Schneid and Lespedeza capitata. The x-axis is time after treatment in hours (h). The y-axis is LUC activity.

FIG. 2 is a graph demonstrating the effects of extracts of Lespedeza bicolor Turcz, Lespedeza cuneata G. Don, Lespedeza maximowiczii C. K. Schneid and Lespedeza capitata, in alleviating reduced biorhythm by environmental hormones. The x-axis is time after treatment in hours (h). The y-axis is LUC activity.

FIG. 3 is a graph of the expression levels of Bmal-1, Per-2 and Cry-1 at 24 hours and 36 hours on synchronized skin explants without application of blue light (left hand graph) and on synchronized skin explants following application of blue light (right hand graph). The y-axis Bmal-1, Per-2 and Cry-1 staining (arbitrary unit).

FIG. 4 is a graph of the expression levels of Bmal-1, Per-2 and Cry-1 at 24 hours and 36 hours on synchronized skin explants following application of blue light to which no Lespedeza capitata extract has been applied (left hand graph) and on following application of Lespedeza capitata extract (right hand graph). The y-axis Bmal-1, Per-2 and Cry-1 staining (arbitrary unit).

FIG. 5.1 is a graph of the expression levels of Nrf2 (y axis indicates Nrf2 staining (arbitrary unit)) on synchronized skin explants alone (A), synchronized skin explants which have been subjected to blue light exposure (B) and synchronized skin explants which have been subjected to blue light exposure and treated with Lespedeza capitata extract (C). FIG. 5.2 is a graph of protein carbonylation (y axis indicates carbonylated proteins staining (arbitrary unit)) on synchronized skin explants alone (A), synchronized skin explants which have been subjected to blue light exposure (B) and synchronized skin explants which have been subjected to blue light exposure and treated with Lespedeza capitata extract (C).

FIG. 6 is a graph of the expression level of Aquaporin-3 (y axis indicates Aquaporin-3 staining (arbitrary unit)) at 24 hours and 36 hours on synchronized skin explants alone (left hand graphs, A), on synchronized skin explants which have been subjected to blue light exposure (middle graphs, B) and on synchronized skin explants which have been subjected to blue light exposure and treated with Lespedeza capitata extract (right-hand graphs, C).

FIG. 7 is a graph of Complexion Index Improvement (%) as measured by a G150 SEELAB® Gonio-Spectrophotometer on the skin of a panel of volunteers which has been treated with a composition comprising Lespedeza capitata extract versus a placebo which comprised water in place of the Lespedeza capitata extract (see Experimental Example 7).

FIG. 8 is a graph of the results of a self-evaluation of the participants' skin from the same panel of volunteers in FIG. 7 (see Experimental Example 7). It measures the percentage of volunteers observing an improvement of skin quality, where A is “complexion is fresh”, B is “complexion is rested”, C is “complexion is more homogenous” and D is “skin is radiant”.

### DESCRIPTION OF EMBODIMENTS

Hereinafter, for better understanding of the present invention, examples will be described in detail. However, the embodiments of the present invention can be modified into various other forms, but the scope of the present invention should not be construed as being limited to the following embodiments. Embodiments of the present invention are provided in order to fully describe the present invention to those skilled in the art.

### Embodiment 1: Preparation of the Extracts of Lespedeza bicolor Turcz, Lespedeza cuneata G. Don and Lespedeza maximowiczii C. K. Schneid

Lespedeza bicolor Turcz, Lespedeza cuneata G. Don and Lespedeza maximowiczii C. K. Schneid, are collected from Jeju Island. In each case, the entire plant from the leaves to the roots was shaved at 20 to 35° C., and then pulverized to make the particle size to be 1 mm or less. Then, 1 kg of pulverized powder was immersed in a 70% ethanol solvent and subjected to ultrasonic extraction for 48 hours at 70° C. and the obtained extract was filtered using a filter paper (Advantes, No. 2). Following this, the filtrate was concentrated under reduced pressure and then the separate the extracts of each of Lespedeza bicolor Turcz, Lespedeza cuneata G. Don and Lespedeza maximowiczii C. K. Schneid, were prepared.

### Embodiment 2: Preparation of the Extracts of Lespedeza capitata

Lespedeza capitata are collected, the entire plant from the leaves to the roots was shaved at 20 to 35° C., and then pulverized to make the particle size to be 1 mm or less. Then, 1 kg of pulverized powder was immersed in a 70% ethanol solvent and subjected to ultrasonic extraction for 48 hours at 70° C. and the obtained extract was filtered using a filter paper (Advantes, No. 2). Following this, the filtrate was concentrated under reduced pressure and then the extracts of Lespedeza capitata were prepared.

### Experimental Example 1: Measurement of the Enhancement Effect for the Circadian Clock Activity by the Extracts of Lespedeza bicolor Turcz, Lespedeza cuneata G. Don, Lespedeza maximowiczii C. K. Schneid and Lespedeza capitata

In order to measure the enhancement effects in skin circadian clock activity by the extracts of Lespedeza bicolor Turcz, Lespedeza cuneata G. Don, Lespedeza maximowiczii C. K. Schneid and Lespedeza capitata, human skin keratinocytes containing Per2pro-LUC (HaCaT) were inoculated in 96-well plate containing Hygromycine B at 200 ug/ml and 10% FBS (Fetal Bovine Serum) in DMEM medium (1.4×104 cell/well), and were cultured in 5% CO2 incubator at 37° C. for 48 hours. 0 and 50 ug/ml of Lespedeza extracts were each treated for 24 hours and the luminescence was measured.

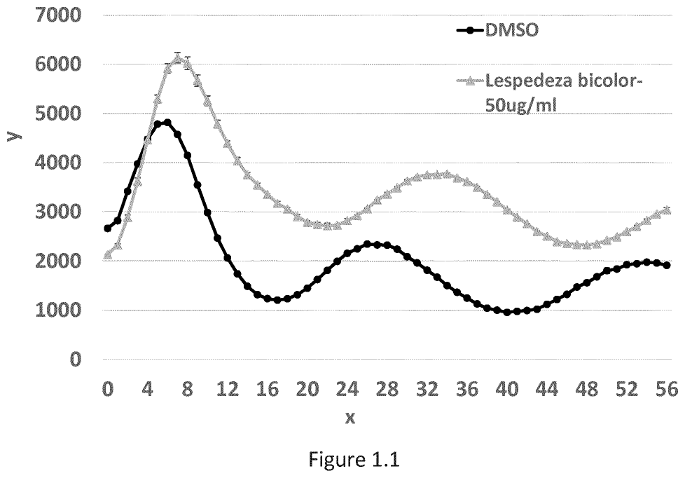

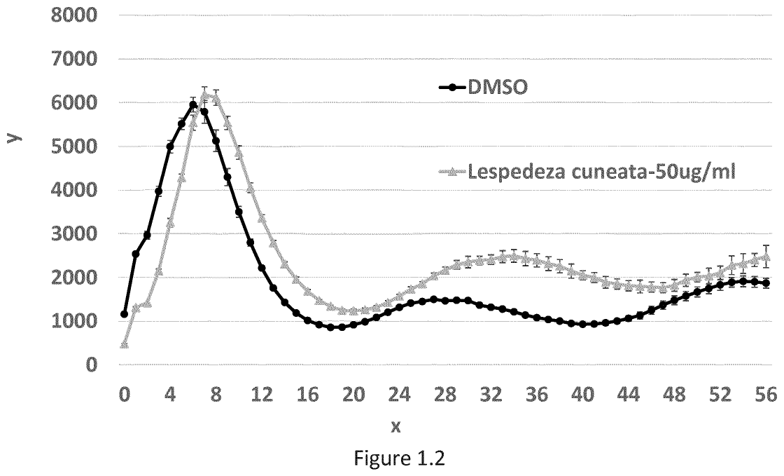

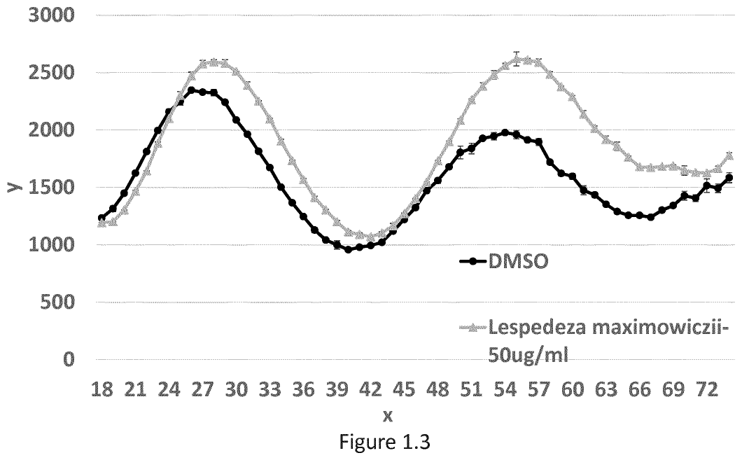

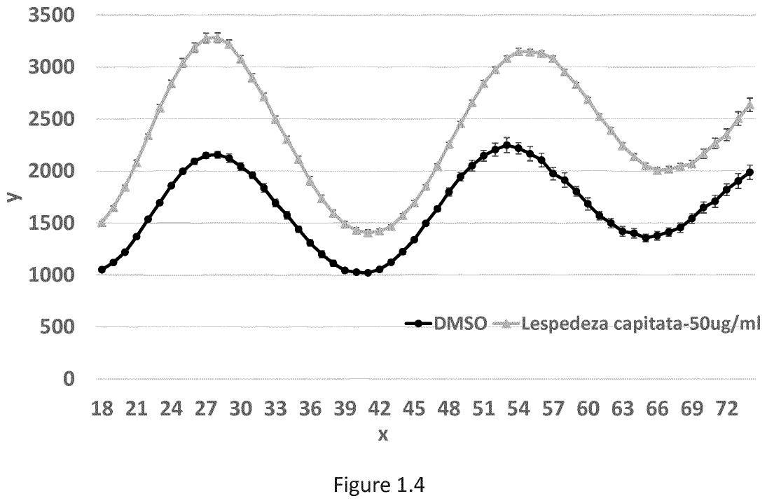

FIGS. 1.1 to 1.4 are graphs demonstrating the biorhythm-enhancing effect of extracts of Lespedeza bicolor Turcz, Lespedeza cuneata G. Don, Lespedeza maximowiczii C. K. Schneid and Lespedeza capitata. As demonstrated here, when the cells are treated with the extracts of Lespedeza bicolor Turcz, Lespedeza cuneata G. Don, Lespedeza maximowiczii C. K. Schneid and Lespedeza capitata, the absolute values of Per2pro-LUC rhythm are all increased.

### Experimental Example 2: Measurement of the Improvement Effect in Circadian Clock Activity Reduced by Environmental Stress

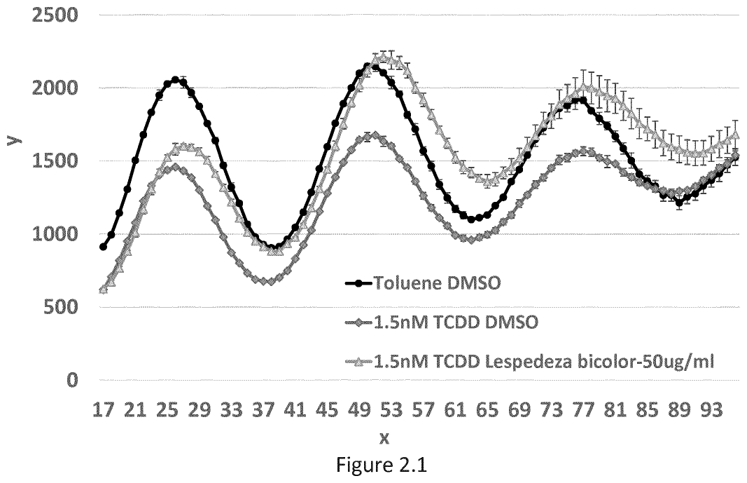

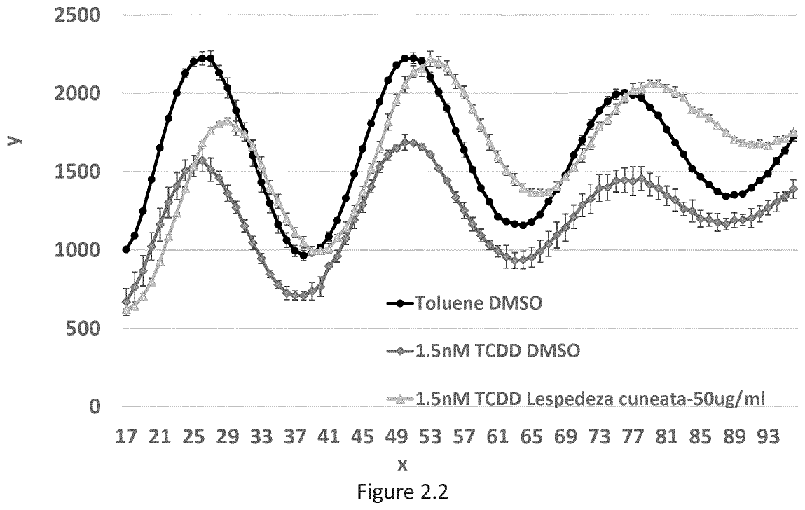

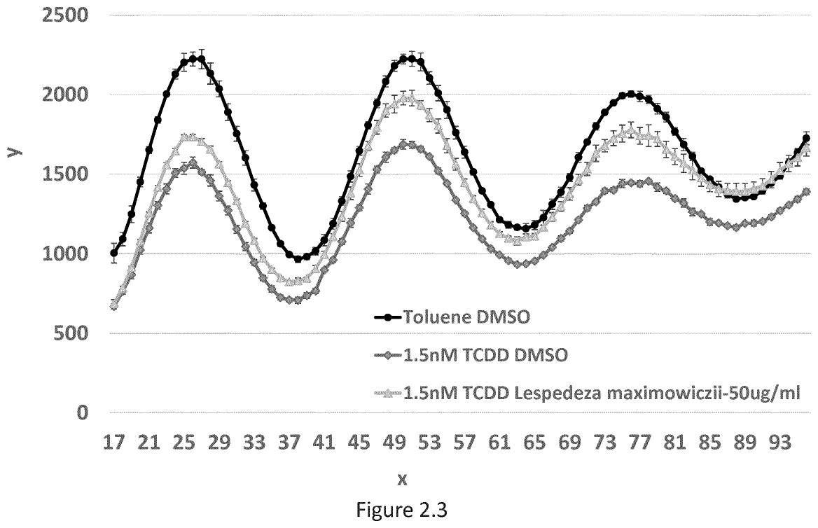

The improvement effects of the extracts of Lespedeza bicolor Turcz, Lespedeza cuneata G. Don, Lespedeza maximowiczii C. K. Schneid and Lespedeza capitata were measured in skin circadian clock activity reduced by TCDD (2,3,7,8-Tetrachlorodibenzo-p-dioxin) which is an environmental hormone dioxin. HaCaT cells containing Per2pro-LUC were inoculated in a 96-well plate containing Hygromycine B at 200 ug/ml and 10% FBS in DMEM medium (1.4×104 cells/well), and cultured in a 5% CO2 incubator at 37° C. for 48 hours. After 48 hours, 50 ug/ml of the extracts of Lespedeza bicolor Turcz, Lespedeza cuneata G. Don and Lespedeza maximowiczii C. K. Schneid and Lespedeza capitata were each treated for 24 hours while being treated with TCDD (2,3,7,8-tetrachlorodibenzo-p-dioxin) 1.5 nM for 4 hours simultaneously, and then transferred to the medium for measuring Luciferase (DMEM containing HEPES 10 mM, Luciferin 1 mM, Hygromycine B 200 ug/ml, 10% FBS). The results are demonstrated in FIGS. 2.1 to 2.4, which are graphs demonstrating the effects of extracts of Lespedeza bicolor Turcz, Lespedeza cuneata G. Don, Lespedeza maximowiczii C. K. Schneid and Lespedeza capitata in alleviating reduced biorhythm by environmental hormones. As shown here, cells treated with TCDD had 30% reduced absolute value for Per2pro-LUC rhythm, compared to cells that were treated with the solvent toluene alone. In contrast to this, cells treated with the extracts of Lespedeza bicolor Turcz, Lespedeza cuneata G. Don, Lespedeza maximowiczii C. K. Schneid and Lespedeza capitata demonstrated similar value for Per2pro-LUC rhythm as in the cells treated with solvent toluene only.

In Experimental Examples 3, 4, 5, 6, and 7, skin explants from a 43-year old Caucasian woman were used. The purpose of the experiments was to study (a) the effect in skin explants of blue light on circadian rhythm markers and to determine if blue light is also able to disrupt skin circadian rhythm disrupted and (b) the effect of Lespedeza capitata extract in this disrupted model. To do this, the skin explants were prepared and some were synchronized with dexamethasone at 5 μM for 1 hour, while some were not.

Some synchronized samples were then treated for 12 hours with the following formulation comprising Lespedeza capitata extract:

| | | | |
|---|---|---|---|
| A | Water | Aqua | 82.60 |
| | Aristoflex Velvet | Polyacrylate | 2.00 |
| | | Crosspolymer-11 | |
| B | Xiameter PMX 0200 fluid | Dimethicone | 1.00 |
| | 50 cst | | |
| | Phenonip ME | Phenoxyethanol (and) | 1.20 |
| | | Methylparabem (and) | |
| | | Ethylparaben | |
| | Fragrance Selenium | Fragrance | 0.10 |
| C | Trisodium Citrate | Trisodium Citrate | 0.10 |
| | Water | Aqua | 10.00 |
| D | Lespedeza capitata | Water (and) | 3.00 |
| | extract of Embodiment 2 | Propanediol (and) | |
| | | Lespedeza Capitata | |
| | | Leaf/Stem Extract | |

The synchronized explants (both those treated with Lespedeza capitata extract and the untreated explants) were then exposed to blue light at 85 J/cm² for 4 hours. The explants which had been treated with Lespedeza capitata extract were then treated for a further 12 hours Lespedeza capitata extract once again. Two samplings took place at 24 and 36 hours after the end of dexamethasone synchronization.

### Experimental Example 3: Measurement of the Effect of Blue Light on Circadian Markers in the Synchronized Model

As discussed above, in a first step, some skin explants were synchronized using dexamethasone to restore normal expression of key circadian markers, Bmal-1, Per-2 and Cry-1. Non-synchronized skin explants show a dysregulated expression of these proteins and a loss of anti-phasic expressions between morning and evening markers, which is restored by the dexamethasone synchronization.

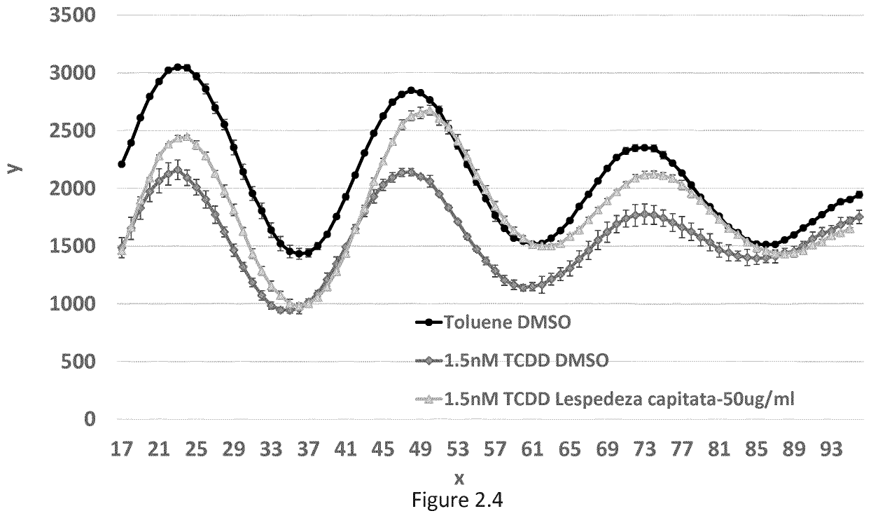

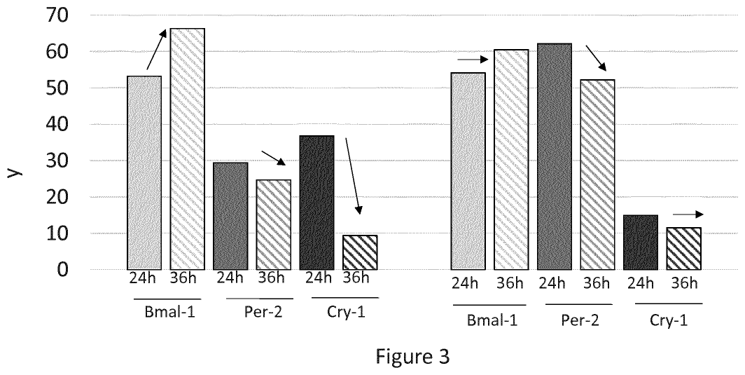

On a dexamethasone synchronized model, we observed that blue light exposure modulates the protein expression levels for Bmal-1 and Cry-1. With reference to FIG. 3, there is no statistical differences in the level of protein expressions from 24 h to 36 h. Blue light increases the level of Per-2 protein expression at 24 h and 36 h post synchronization in comparison to dexamethasone control, but didn't alter the phase for Per-2 marker as we still observe a difference in the level expression between 24 h and 36 h.

This experiment demonstrates that blue light exposure dysregulates significantly skin circadian cycle for 3 key markers.

### Experimental Example 4: Treatment by Lespedeza capitata Extract to Improve the Circadian Rhythm in Synchronized Skin Explants which have been Stressed by Exposure to Blue Light

In Experimental Example 3, it was demonstrated that blue light disrupts circadian cycle. In contrast, it was observed that application of the Lespedeza capitata extract of Embodiment 2 counteracts the disruption caused by blue light. The Lespedeza capitata extract restores the phase rhythmicity, and improves the amplitude of the circadian cycle, for all markers.

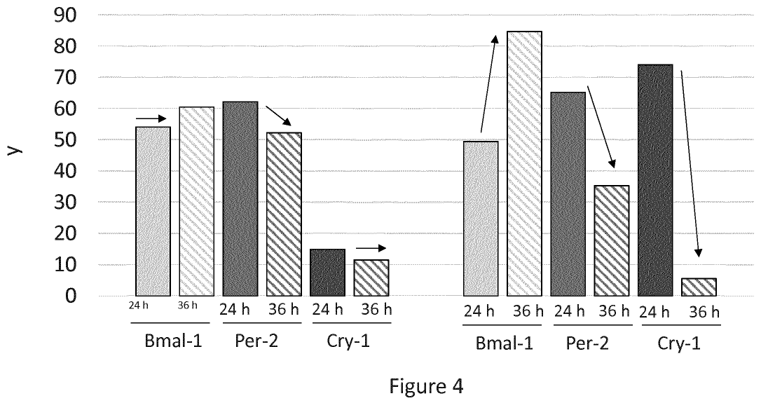

With reference to FIG. 4, during the period from 24 hours to 36 hours, a strong increase in Bmal-1 expression was observed; for Per-2 and Cry-1 strong decreases were observed.

Statistical analysis shows that the Lespedeza capitata extract increases Bmal-1 protein expression at 36 hours in comparison to 24 hours.

In comparison to blue light stress condition alone at 36 hours, B-Lespedeza capitata extract increases significantly Bmal-1 protein expression.

Lespedeza capitata extract significantly increases Cry-1 protein expression at 24 hours in comparison to blue light stress condition alone. This phenomenon leads to a significant increase of Cry-1 expression amplitude during the period from 24 hours to 36 hours after synchronization. Quantification shows that Cry-1 expression at 36 h is close to statistical difference in Lespedeza capitata extract condition compared the blue light condition. The data demonstrates also an increase rhythmicity for Per-2 expression with a statistical difference between Lespedeza capitata extract condition and the blue light stress condition alone at 36 hours.

### Experimental Example 5: Stimulation of Detoxification Pathway Under Blue Light on Synchronized Skin Ex Vivo: Nrf-2 and Carbonylated Proteins Staining Quantification

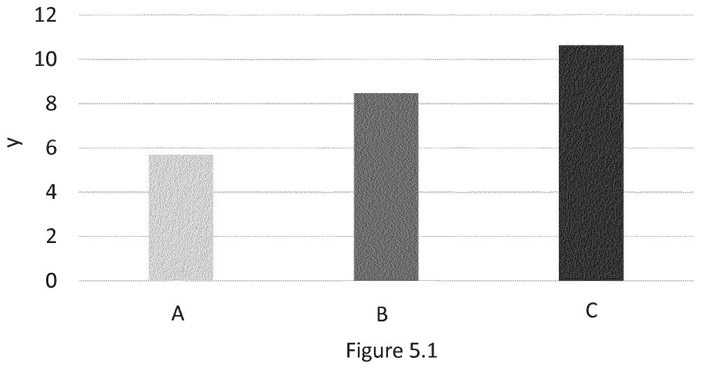

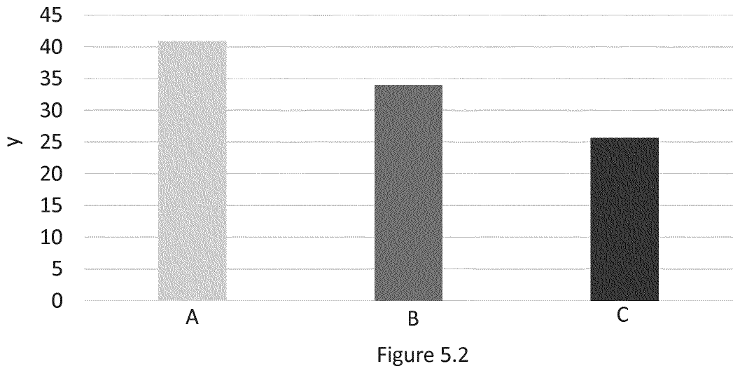

With reference to FIGS. 5.1 and 5.2, it was observed that in this synchronized model, under application of blue light, that Lespedeza capitata extract significantly stimulates Nrf-2 protein expression. Concomitantly, our results show that Lespedeza capitata extract reduces the protein carbonylation, a consequence of high ROS content, produced by blue light exposure, suggesting a better anti-oxidant protection. These results confirm that when cells are synchronized, a better detoxification activity is visible, as confirmed by the increase nrf-2 expression and less carbonylated of protein.

### Experimental Example 6: Study of Skin Biological Function on Synchronized Skin Ex Vivo: Aquaporin-3

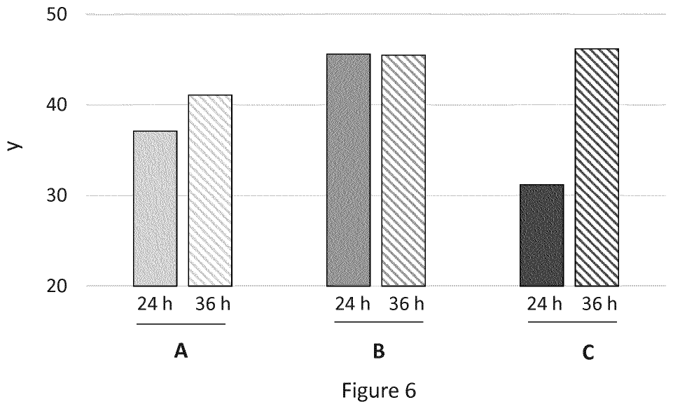

A certain number of proteins is known to follow a circadian rhythm, like aquaporin-3. Our synchronized model confirms the existence of a time-dependent skin hydration by AQP-3. Blue light stress induces a dysregulation in AQP-3 expression which is not time-dependent, as it should be. Indeed, blue light stress significantly increases AQP-3 expression at 24 hours, leading to a loss of rhythmicity. With reference to FIG. 6, Lespedeza capitata extract treatment gives rise to a significant difference at 24 hours leading to a significant increase of AQP-3 at 36 hours. Lespedeza capitata extract restores and improves the rhythmicity of Aquaporin-3 expression in human skin.

### Experimental Example 7: Clinical Study Performed on a Panel of Volunteers

The aim of the clinical study was to demonstrate the beneficial effect of Lespedeza capitata extract on the skin's well-being by studying the skin complexion and fatigue parameters.

The double blinded clinical study involved 17 volunteers having a disrupted circadian rhythm. Shift workers were selected, because the nature of this work gives rise to a disrupted circadian rhythm. The volunteers were aged between 35 and 55 (average age: 43)

The volunteers were asked to apply to one half of their face the following cream containing 3% Lespedeza capitata extract twice daily for 28 days:

| | | | |
|---|---|---|---|
| A | Water | Aqua | 82.60 |
| | Aristoflex Velvet | Polyacrylate | 2.00 |
| | | Crosspolymer-11 | |
| B | Xiameter PMX 0200 fluid | Dimethicone | 1.00 |
| | 50 cst | | |
| | Phenonip ME | Phenoxyethanol (and) | 1.20 |
| | | Methylparabem (and) | |
| | | Ethylparaben | |
| | Fragrance Selenium | Fragrance | 0.10 |
| C | Trisodium Citrate | Trisodium Citrate | 0.10 |
| | Water | Aqua | 10.00 |
| D | Lespedeza capitata | Water (and) | 3.00 |
| | extract of Embodiment 2 | Propanediol (and) | |
| | | Lespedeza Capitata | |
| | | Leaf/Stem Extract | |

A placebo was applied in the other half face. The placebo formulation was identical to the above formulation, except that part D was replaced with 3 wt % water. Measurements were done after one week and after four weeks of treatment.

A dull complexion is the key visual parameter associated with a deregulated circadian cycle, so the skin complexion was determined by the gonio-spectrophotometer (the device was a GP150 made by SEELAB®). Moreover, volunteers were asked to answer to a questionnaire related to their perception of their skin quality.

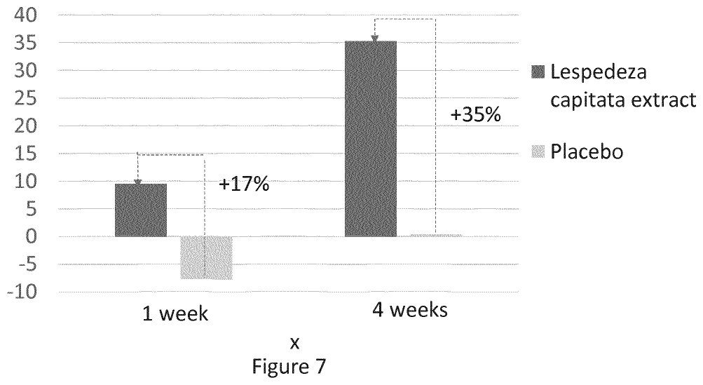

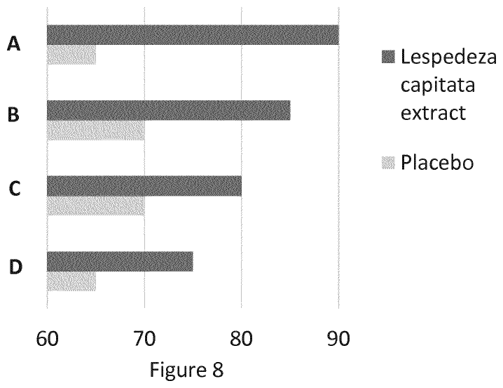

With reference to FIG. 7, after 4 weeks, application of the cream containing 3 wt % Lespedeza capitata extract improved the skin's complexion by +35% as measured by the SEELAB® gonio-spectrophotometer, meaning the complexion is more radiant, homogeneous and has improved skin tone. One of the main parameters induced by the circadian rhythm disruption is controlled. An increase in the complexion index was also observed, which corresponds to an improved of skin complexion. In addition, volunteers observed a significant positive effect of Lespedeza capitata extract on their complexion and skin radiance, which are parameters associated with the skin's well-being (see FIG. 8).

### Formulation Example 1: Cosmetic Formulation

1-1. Softening Toilet Water

TABLE 1

| Ingredients | Content % |
|---|---|
| Lespedeza bicolor Turcz extracts | 0.05 |
| glycerin | 3.0 |
| butylene glycol | 2.0 |
| propylene glycol | 2.0 |
| Carboxyvinyl polymer | 0.1 |
| Ethanol | 10.0 |
| Triethanolamine | 0.1 |
| Preservative, trace pigment, trace flavour and | 82.74 |
| trace amount of purified water | |
| Total | 100.0 |

1-2. Nourishing Toilet Water

TABLE 2

| Ingredients | Content % |
|---|---|
| Lespedeza bicolor Turcz extracts | 0.05 |
| Wax | 4.0 |
| Polysorbate 60 | 1.5 |
| Sorbitan sesquioleate | 0.5 |
| Liquid paraffin | 5.0 |
| Squalane | 5.0 |
| Kaprilic/Capric triglyceride | 5.0 |
| Glycerin | 3.0 |
| Butylene glycol | 3.0 |
| Propylene glycol | 3.0 |
| Carboxyvinyl polymer | 0.1 |
| Triethanolamine | 0.2 |
| Preservative, trace pigment, trace flavour and | 69.64 |
| trace amount of purified water | |
| Total | 100.0 |

1-3. Nourishing Cream

TABLE 3

| Ingredients | Content % |
|---|---|
| Lespedeza capitata extract, made according to | 0.05 |
| Embodiment 2 | |
| Wax | 10.0 |
| Polysorbate 60 | 1.5 |
| Sorbitan sesquioleate | 0.5 |
| Liquid paraffin | 10.0 |
| Squalane | 5.0 |
| Kaprilic/Capric triglyceride | 5.0 |
| Glycerin | 5.0 |
| Butylene glycol | 3.0 |
| Propylene glycol | 3.0 |
| Triethanolamine | 0.2 |
| Preservative, trace pigment, trace flavour and | 56.74 |
| trace amount of purified water | |
| Total | 100.0 |

1-4. Massage Cream

TABLE 4

| Ingredients | Content % |
|---|---|
| Lespedeza bicolor Turcz extracts | 0.05 |
| Wax | 10.0 |
| Polysorbate 60 | 1.5 |
| Sorbitan sesquioleate | 0.8 |
| Liquid paraffin | 40.0 |
| Squalane | 5.0 |
| Kaprilic/Capric triglyceride | 4.0 |
| Glycerin | 5.0 |
| Butylene glycol | 3.0 |
| Propylene glycol | 3.0 |
| Triethanolamine | 0.2 |
| Preservative, trace pigment, trace flavour and | 27.45 |
| trace amount of purified water | |
| Total | 100.0 |

2-5. Face Mask

TABLE 5

| Ingredients | Content % |
|---|---|
| Lespedeza bicolor Turcz extracts | 0.05 |
| Polyvinyl alcohol | 13.0 |
| Sodium carboxymethyl cellulose | 0.2 |
| Allantoin | 0.1 |
| Ethanol | 5.0 |
| Nonylphenyl ether | 0.3 |
| Preservative, trace pigment, trace flavour and | 81.34 |
| trace amount of purified water | |
| Total | 100.0 |

### Formulation Example 2: Pharmaceutical Formulation

2-1. Manufacture of Powder

TABLE 6

| Ingredients | g |
|---|---|
| Lespedeza bicolor Turcz extracts | 2 |
| Lactose | 1 |

The above components were mixed and filled in airtight bags to prepare powders.

2-2. Manufacture of Tablets

TABLE 7

| Ingredients | mg |
|---|---|
| Lespedeza bicolor Turcz extracts | 100 |
| Corn starch | 100 |
| Lactose | 100 |
| Magnesium stearate | 2 |

After mixing the above ingredients, tablets were prepared by tableting the ingredients according to a conventional method for producing tablets.

2-3. Preparation of Capsules

TABLE 8

| Ingredients | mg |
|---|---|
| Lespedeza bicolor Turcz extracts | 100 |
| Corn starch | 100 |
| Lactose | 100 |
| Magnesium stearate | 2 |

After mixing the above ingredients, the ingredients were then filled into gelatin capsules according to the conventional method for preparing capsules.

1.-6. (canceled)

7. A method of preventing or alleviating circadian rhythm disorder in a mammal, the method comprising administering to a mammal in need thereof a composition comprising at least one extract from a Lespedeza sp. plant.

8. A method of preventing or alleviating circadian rhythm disorder in mammalian skin, the method comprising administering to the skin of a mammal in need thereof a composition comprising at least one extract from a Lespedeza sp. plant.

9. The method of claim 7, wherein the Lespedeza sp. plant is at least one Lespedeza sp. plant selected from a group consisting of Lespedeza capitata, Lespedeza bicolor Turcz, Lespedeza cuneata G. Don, and Lespedeza maximowiczii C. K. Schneid.

10. The method of according to claim 7, wherein the Lespedeza sp. plant is Lespedeza capitata.

11. The method according to claim 7, wherein the composition comprises from 0.00001 to 15% by weight, of the extract from at least one Lespedeza sp. plant relative to the total weight of the composition.

12. The method according to claim 7, wherein the circadian rhythm disorder is caused by environmental stress.

13. A cosmetic, non-therapeutic treatment of the skin, comprising at least one extract from at least one Lespedeza sp. plant.

14. (canceled)

15. (canceled)

16. The cosmetic, non-therapeutic treatment of the skin according to claim 13, wherein the Lespedeza sp. plant is at least one plant selected from a group consisting of Lespedeza capitata, Lespedeza bicolor Turcz, Lespedeza cuneata G. Don, and Lespedeza maximowiczii C. K. Schneid.

17. The cosmetic, non-therapeutic treatment to increase the radiance of the skin according to claim 13, wherein the Lespedeza sp. plant is Lespedeza capitata.

18. The cosmetic, non-therapeutic treatment to increase the radiance of the skin according to claim 13, wherein the composition comprises from 0.00001% to 15% by weight, of the extract from a Lespedeza sp. plant relative to the total weight of the composition.

19. The method of claim 8, wherein the Lespedeza sp. plant is at least one Lespedeza sp. plant selected from a group consisting of Lespedeza capitata, Lespedeza bicolor Turcz, Lespedeza cuneata G. Don, and Lespedeza maximowiczii C. K. Schneid.

20. The method of according to claim 8, wherein the Lespedeza sp. plant is Lespedeza capitata.

21. The cosmetic, non-therapeutic treatment to increase the complexion of the skin according to claim 14, wherein the Lespedeza sp. plant is Lespedeza capitate.

22. The cosmetic, non-therapeutic treatment to enhance skin biorhythms according to claim 15, wherein the Lespedeza sp. plant is Lespedeza capitate.

23. The cosmetic, non-therapeutic treatment of the skin according to claim 13, wherein the cosmetic, non-therapeutic treatment increases the radiance of the skin, improves the complexion of the skin, and/or enhances the skin biorhythms.
